(19) United States
(12) Patent Application Publication
(10) Pub. No.: US 2019/0321481 A1
(43) Pub. Date: Oct. 24, 2019

(54) IMMUNOMODULATORY COMPOSITIONS, PROCESSES FOR MAKING THE SAME, AND METHODS FOR INHIBITING CYTOKINE STORMS

(71) Applicants: NantBio, Inc., Culver City, CA (US); Nant Holdings IP, LLC, Culver City, CA (US)

(72) Inventors: Kayvan NIAZI, Encino, CA (US); Nicholas J. WITCHEY, Culver City, CA (US)

(73) Assignees: NantBio, Inc., Culver City, CA (US); Nant Holdings IP, LLC, Culver City, CA (US)

(21) Appl. No.: 16/348,696

(22) PCT Filed: Nov. 9, 2017
(86) PCT No.: PCT/US2017/060865

§ 371 Date: May 9, 2019

Related U.S. Application Data
(60) Provisional application No. 62/420,763, filed on Nov. 11, 2016.

(51) Int. Cl. A61K 47/64; A61K 47/65; C07K 16/28; A61K 39/35; A61K 39/145; A61K 39/215; A61K 39/02; A61K 39/285; A61K 39/395; A61K 39/00

(52) U.S. Cl. CPC A61K 47/646; A61K 47/65; C07K 16/2827; A61K 39/35; A61K 39/145; A61K 39/215; C07K 2317/34; A61K 39/285; A61K 39/39558; A61K 39/00119; A61K 2039/505; A61K 2039/876; A61K 2039/627; A61K 39/02

(57) ABSTRACT

A chimeric immune modulator is provided, that comprises a first immune modulating portion and a second portion, wherein the second portion comprises an epitope; and the modulating portion and the second portion of the moiety are coupled together via a linker. Methods of making and using the chimeric immune modulator are also provided. Methods of making and using the chimeric immune modulator are also provided.

### FIELD OF THE INVENTION

The field of the invention relates to chimeric immune modulators for inhibiting or downregulating cytokine storms caused by identified epitopes.

### BACKGROUND OF THE INVENTION

The background description includes information that may be useful in understanding the present invention. It is not an admission that any of the information provided herein is prior art or relevant to the presently-claimed invention, or that any publication specifically or implicitly referenced is prior art.

All publications, patents, and patent applications recited herein are incorporated by reference to the same extent as if each individual publication, patent, or patent application were specifically and individually incorporated by reference. Where a definition or use of a term in an incorporated publication, patent, or patent application is inconsistent or contrary to the definition of that term provided herein, the definition of that term provided herein applies and the definition of that term in the reference does not apply.

In humans and other vertebrate subjects, a successful immune response to certain diseases or injuries, e.g., a microbial or viral infection, exposure to some other type of antigen, or other form of injury, typically produces an increase in pro-inflammatory cytokines and related factors. Depending on the nature of the inciting event, this can result, for example in fever, chills, localized swelling, and redness in an affected tissue or organ. Generally, this proinflammatory reaction subsides as the infection or other antigen(s) is cleared, or as the injury heals. However, for reasons that are not well understood, in certain subjects, the reaction of the immune system becomes an over-reaction driven by a “positive” proinflammatory feedback loop between cytokines and immune system cells. This proinflammatory loop can result from infection by viral diseases such as the H1N1 strain of influenza, Ebola and others, and/or from contact by a subject with other types of antigens, such as toxins produced by S. aureus. In other cases, the initiating factor of the proinflammatory loop can be obscure, e.g., an experimental drug. If the proinflammatory reaction is extreme, the result is considered to be a “cytokine cascade,” also referred to as hypercytokinemia, or more commonly described as a “cytokine storm.” The surge in proinflammatory cytokines and other factors induces dangerously elevated fever, a precipitous drop in blood pressure leading to shock, and can produce organ specific damage, e.g., to the lung or other tissues. In this circumstance, a proinflammatory reaction may become so severe that it becomes life-threatening. See, for example, Tisonscik et al., 2012, Microbiol Mol Biol Rev. 76(1): 16-32; doi: 10.1128/MMBR.05015-11). To date, there are no satisfactory or reliable methods of preventing or treating a cytokine storm in subjects experiencing this reaction, particularly when the cytokine storm results from cancer immunotherapy, autoantigens and/or antigens found in foods or other ingestible materials.

Immunotherapy broadly refers to any form of treatment that causes a subject's immune system to treat or inhibit a disease or disorder. Anti-cancer immunotherapy encompasses a number of different ways to harness the immune response to treat cancer in a subject. For example, cell growth checkpoint inhibitors, such as Yervoy® (ipilimumab) and Opdivo® (nivolumab), made by Bristol-Myers Squibb; Keytruda® (pembrolizumab), by Merck; and Tecentriq® (atezolizumab), by Genentech are known. Cell therapy such as collecting autologous immune cells from a subject with cancer, modifying the harvested cells ex vivo so that they will target the subject's cancer, and then reinfusing the modified, harvested cells back into the subject are known.

Vaccines engineered to elicit an immune response against a subject specific and/or tumor specific antigen, or neoantigen, e.g., as described by US20140178438, US20160008447 and/or by U.S. provisional patent application Ser. No. 62/373,486, filed on Aug. 11, 2016, are also known, While individualized anticancer therapy is increasingly favored, with such therapy comes the risk of idiosyncratic reactions such as cytokine storms.

In particular, there is a concern that vaccines designed to elicit an immune response to an antigen specific for a particular subject, and/or specific to the cancer of a particular subject, i.e., a neoepitope, might produce a life-threatening cytokine storm. Thus, there is a need in the art for improved medications and methods for preventing and/or downregulating a cytokine storm, in a subject.

### SUMMARY OF THE INVENTION

The invention is directed to various compositions, methods, and uses of therapeutic modalities in immune therapy. According to the present invention, there is provided a chimeric immune modulator, and methods of making and using the same. In a first embodiment, the chimeric immune modulator comprising a first portion, that is an immune modulator, and a second portion, wherein the second portion comprises an epitope; and the modulating portion and the second portion of the moiety are coupled together, optionally via a linker. The epitope is an identified epitope, such as a neoepitope, an autoantigen and/or an exogenous antigen. The connecting linker is a peptide, a polymer, or a covalent bond that does not significantly interfere with the function of the first and second portion of the chimeric immune modulator.

In another aspect, the immune modulating portion of the chimeric immune modulator includes a ligand for an immune checkpoint inhibition receptor. This is, for example, one or more of a CTLA4 ligand, a PD1 ligand, and/or a B7-H4 molecule. In a further embodiment, the modulating portion includes a negative checkpoint regulator. The negative checkpoint regulator is contemplated to include one or more of a VISTA molecule, an inhibitor of a co-stimulatory molecule. The co-stimulatory molecule is, for example, one or more of CD80 and/or CD86.

In a further aspect, the chimeric immune modulator includes multiple distinct epitopes and/or multiple distinct immune modulators. For example, in certain embodiments, the chimeric immune modulator includes at least two of a ligand for an immune checkpoint inhibition receptor, a negative checkpoint regulator, and/or an inhibitor of a co-stimulator molecule.

Preferably, the epitope portion of the chimeric immune modulator is patient-specific, and/or, is tumor-specific. In certain preferred aspects of the invention, the epitope is an HLA-matched neoepitope.

In a second embodiment, the invention provides a method of modulating an over-reaction of a subject's immune system to an epitope, the method comprising administering an effective amount of the inventive chimeric immune modulator to a subject. The chimeric immune modulator is optionally administered to the subject topically, intravenously, subcutaneously, and/or orally. The epitope is contemplated to include a cancer neoepitope, an autoantigen and/or an exogenous antigen, such as a food antigen, e.g., gliadin, a gluten, and a peanut allergen.

In one aspect, the over-reaction of the immune system is contemplated to be associated with an MHC-II polymorphism, e.g., that results in one or more of Type-I diabetes, rheumatoid arthritis, celiac disease, and/or pemphigus vulgaris. In another aspect, the over-reaction of the immune system is contemplated to be associated with an MHC-I polymorphism, e.g., that results in one or more of Hashimoto's Thyroiditis, systemic lupus erythematodes, and/or blepharitis.

In another aspect of the method of the invention, it is contemplated that the method further includes administering an antibody that binds to the epitope that triggered the over-reaction. The antibody is, for example, an immunoglobulin that does not activate a cellular immune response, such as an antibody that includes an IgG4 portion. In a further aspect, the method also optionally includes a step of administering a therapeutically active molecule that inhibits or downregulates a system that delivers the epitope to the subject. The therapeutically active molecule is, for example, an antibody that binds to a virus that delivers the epitope to the subject. In yet a further aspect, the method includes administering an effective amount of an immunosuppressive drug. The immunosuppressive drug is contemplated to include, for example, one or more of a steroid, ribavirin, an anti-MHC II antibody, an anti-cytokine antibody, cyclosporine, and cyclophosphamide.

The effective amount of the chimeric immune modulator is contemplated to be administered to the subject according to any effective art known schedule. The schedule of administration can include, for example, a metronomic schedule. The metronomic schedule is, for example, based on a time schedule and/or based on severity of the over-reaction of the subject's immune system, or alternatively, the metronomic schedule is based on a concentration of the administered immune modulator.

In a third embodiment, the inventive method includes measuring a metric of an immune response to a treatment associated with the chimeric immune modulator. In certain aspects, the metric includes at least one of:
a) a measurement of at least one cytokine;
b) a measurement of a T-cell concentration;
c) a measurement of a T-cell frequency;
d) an ELISA measurement of B-cell activation; and
e) a measurement of T-cell proliferation.

The inventive treatment method further includes a step of determining an intervention point based on the metric.

In a fourth embodiment, the invention provides for a method of treating cancer in a subject diagnosed with a cancer comprising:
a) administering an amount of a neoepitope-based anticancer therapy having an amount of a neoepitope effective to stimulate an immune response to the subject; and
b) administering an effective amount of the inventive chimeric immune modulator to the subject, wherein the epitope of the chimeric immune modulator is the neoepitope of a), and wherein steps a) and b) are conducted simultaneously, or one before the other.

In a further aspect, the inventive neoepitope-based therapy of a) above comprises administering a vaccine to the subject needing anticancer therapy, wherein the vaccine comprises a nucleic acid that encodes the neoepitope, a protein-based vaccine comprising the neoepitope, a cell-based vaccine that presents the neoepitope the immune system of the subject, and/or a viral expression system-based vaccine that presents the neoepitope to the immune system of the subject. The chimeric immune modulator is optionally administered to the subject topically, intravenously, subcutaneously, or orally.

In a fifth embodiment, the invention contemplates a method of preparing the chimeric immune modulator, by the steps of covalently linking one or more epitopes that cause or may cause an over-reaction of a subject's immune system with one or more immune modulators that will downregulate a cytokine storm. The one or more epitopes can be, for example, a cancer neoepitope employed in an anticancer vaccine, an autoantigen and/or an exogenous antigen. In another aspect, the method of making the inventive chimeric immune modulator includes the steps of encoding the one or more epitopes and the one or more immune modulators in a nucleic acid expression vector, and inserting that vector into a suitable host cell by transfection or transformation, and culturing the host cell to produce the chimeric immune modulator.

### DETAILED DESCRIPTION

Accordingly, the invention provides a chimeric immune modulator comprising a first immune modulating portion or immune modulator molecule, and a second portion, wherein the second portion comprises an epitope; and the immune modulating portion and the epitope are coupled together, optionally via a linker. The invention also provides methods of making and using the same.

In order to appreciate the present invention, the following terms are defined. Unless otherwise indicated, the terms listed below will be used and are intended to be defined as stated, unless otherwise indicated. Definitions for other terms can occur throughout the specification.

It is intended that all singular terms also encompass the plural, active tense and past tense forms of a term, unless otherwise indicated.

The phrase “consisting essentially of” means that the composition and methods may include additional ingredients and/or steps, but only if the additional ingredients and/or steps do not materially alter the basic and novel characteristics of the claimed composition or method, i.e., the additional ingredient and/or step(s) would serve no purpose material to the claimed composition or methods.

The terms “subject” and “patient” are used interchangeably throughout. A “subject” or “patient” according to the invention is a vertebrate such as a mammal exhibiting the signs of an over-reaction of the immune system, i.e., a cytokine storm. Preferably, the subject is a human patient, although, in certain embodiments, the invention can also be applied in a veterinary practice to any vertebrate animal in need of such treatment. This could include, for example, mammals such as non-human primates, canines, felines, porcines, equines, and any other animal for which it is desired to treat or prevent a cytokine storm.

An immune modulator, or an immune modulator molecule, is a molecule that will downregulate an immune response, and that usefully inhibits or moderates the extreme immunological reaction of a cytokine storm. Such an immune modulator molecule includes, for example, cytokines that inhibit one or more cellular immune responses. Non-limiting examples of these cytokines include IL-4, IL-10, IL-11, IL-12, and IL-17. An immune modulator can also include, for example, a ligand for an immune checkpoint inhibition receptor, including, e.g., a CTLA4 ligand, a PD1 ligand, and/or a B7-H4 molecule. An immune modulator can also be a negative checkpoint regulator (NCR) that tempers T-cell activation and/or renders cell-mediated immune responses within constraints that are safe to the host subject. Examples of NCRs include VISTA molecules. VISTA molecules are V-domain immunoglobulin (Ig)-containing suppressors of T-cell activation (VISTA). The VISTA NCR is predominantly expressed on hematopoietic cells. Lines et al., 2014, Cancer Immunol Res; 2(6):510-7. doi: 10.1158/2326-6066.CIR-14-0072. The immune modulator is also contemplated to include an inhibitor of a co-stimulatory molecule, such as one or more of CD80, and CD86. Inhibitors of CD80 and/or CD86 include, for example, CTLA-4, CTLA4-Ig, CD28, CD28Ig, and ICOS.

An inventive chimeric immune modulator is a molecule engineered by linkage of one or more of the immune modulator molecules to an identified epitope or epitopes. The chimeric immune modulator optionally includes multiple distinct epitopes. In certain embodiments, a chimeric immune modulator according to the invention is contemplated to include at least 2 immune modulator molecules, or is contemplated to include from 1 to 10 immune modulator molecules, or 10 or more immune modulator molecules, i.e., a complex of immune modulator molecules.

Without meaning to be bound by any theory or hypothesis of the invention, it is contemplated that by linking a triggering epitope to an immune modulator, the resulting chimeric immune modulator more selectively targets the specific immune cells that are participating in a positive feedback loop resulting in a cytokine storm. This selective targeting is more effective in controlling the over-reaction of the immune system, with fewer unintended side effects.

The identified epitope is one for which there is knowledge, or a concern that, a percentage of subjects to which the epitope is administered are at risk for developing an immunological over-reaction, such as a cytokine storm. The epitope to be targeted by the present invention is a specific, identified epitope that is, for example, a cancer neoepitope, an autoantigen, and/or an exogenous antigen.

The inventive subject matter further provides for detecting an over-reaction of a subject's immune system in reaction to an epitope, e.g., a reaction to neoepitope-based anticancer therapy.

### Identification of Cancer Neoepitopes

A cancer neoepitope is, for example, one or more of a cancer-associated or cancer-specific peptide, a nucleic acid, a lipid, a sugar, and/or other type of molecule that the subject reacts to, and in certain subjects, the reaction becomes an extreme and life threatening cytokine storm. A cancer neo-epitope is also a combination of a cancer-associated or cancer-specific peptide, a nucleic acid, a lipid and/or a sugar. In certain embodiments, a cancer neoepitope is a cancer-associated or cancer-specific peptide, protein and/or nucleic acid identified by a genetic comparison between a cancer cell and a normal cell of a subject.

The neoepitope is contemplated to be patient-specific or patient- and tumor-specific. In another embodiment, the neoepitope is an HLA-matched neoepitope. For example, it is contemplated that prior to cancer treatment, a tumor biopsy is obtained from a subject and omics analysis is performed on the obtained sample. In general, it is contemplated that the omics analysis includes whole genome and/or exome sequencing, RNA sequencing and/or quantification, and/or proteomics analysis. Among other options, it is contemplated that genomic analysis can be performed by any number of analytic methods, however, especially preferred analytic methods include whole genome sequencing (WGS) and exome sequencing of both tumor and matched normal sample. Likewise, the computational analysis of the sequence data may be performed in numerous ways. In most preferred methods, however, analysis is performed in silico by location-guided synchronous alignment of tumor and normal samples as, for example, disclosed in US 2012/0059670A1 and US 2012/0066001A1 using BAM files and BAM servers. Of course, alternative file formats (e.g., SAM, GAR, FASTA, etc.) are also expressly contemplated herein.

In addition, RNA sequencing and/or quantification can be performed by any methods known in the art, and may use various forms of RNA. For example, preferred materials include mRNA and primary transcripts (hnRNA), and RNA sequence information may be obtained from reverse transcribed polyAtRNA, which is in turn obtained from a tumor sample and a matched normal (healthy) sample of the same patient. Likewise, it should be noted that while polyA⁺-RNA is typically preferred as a representation of the transcriptome, other forms of RNA (hn-RNA, non-polyadenylated RNA, siRNA, miRNA, etc.) are also deemed suitable for use herein. Preferred methods include quantitative RNA (hnRNA or mRNA) analysis and/or quantitative proteomics analysis. Most typically, RNA quantification and sequencing is performed using qPCR and/or rtPCR based methods, although other methods (e.g., solid phase hybridization-based methods) are also deemed suitable. Viewed from another perspective, transcriptomic analysis may be suitable (alone or in combination with genomic analysis) to identify and quantify genes having a cancer and patient specific mutation.

Similarly, proteomics analysis can be performed by numerous methods, and all known methods of proteomics analysis are contemplated herein. However, particularly preferred proteomics methods include antibody-based methods and mass spectroscopic methods (and especially selected reaction monitoring). Moreover, it should be noted that the proteomics analysis may not only provide qualitative or quantitative information about the protein per se, but may also include protein activity data where the protein has catalytic or other functional activity. Exemplary techniques for conducting proteomic assays are described by U.S. Pat. No. 7,473,532 and U.S. Pat. No. 9,091,651.

Neoepitopes as used herein are characterized as random mutations or pattern-type mutations in tumor cells that give rise to unique and tumor specific antigens. As such, it should be noted that exome and/or high-throughput genome sequencing allows for rapid and specific identification of patient or subject specific neoepitopes, particularly where the analysis also takes into account matched normal tissue of the same subject.

Therefore, candidate tumor-specific neoepitopes are identified against a matched normal sample of a patient, Additional filtering criteria are contemplated to include:
Does the candidate tumor specific epitope represent a changed peptide sequence?

Is the candidate tumor specific epitope expressed in the tumor? The candidate tumor specific epitope is included in the analysis only if the neoepitope is due to a missense mutation and/or above a minimum expression level (e.g., at least 20%).

If expressed, will the candidate tumor specific epitope bind to the target?
The candidate tumor specific epitope is not used if it is also expressed elsewhere in the patient, to avoid off target immune reactions.

Additionally, such filtering can be further refined by confirming high transmembraneous expression level of cancer neoepitopes. To facilitate computational analysis, it is further contemplated that neoepitopes will be confined to relatively small fragments having a minimum size necessary for antibody binding (e.g., at least 5-6 amino acids) and a maximum size of 20 amino acids (and in some cases longer). Thus, suitable neoepitopes will preferably have a length of between 7-12 amino acids, for example, nine amino acids, including the changed amino acid.

Genomic analysis can be performed by any number of analytic methods. However, especially preferred analytic methods include exome and whole genome sequencing of both tumor and matched normal sample. Likewise, the computational analysis of the sequence data may be performed by numerous methods. However, in particularly preferred methods, analysis is performed in silico by location-guided synchronous alignment of tumor and normal samples as, for example, disclosed in US 2012/0059670A1 and US 2012/0066001A1 using BAM files and BAM servers. It should be noted that any language directed to a “computer” should be read to include any suitable combination of computing devices, including servers, interfaces, systems, databases, agents, peers, engines, controllers, or other types of computing devices operating individually or collectively. One should appreciate that the computing devices comprise a processor configured to execute software instructions stored on a tangible, non-transitory computer readable storage medium (e.g., hard drive, solid state drive, RAM, flash, ROM, etc.).

The software instructions preferably configure the computing device to provide the roles, responsibilities, or other functionality as discussed below with respect to the disclosed apparatus. Further, the disclosed technologies can be embodied as a computer program product that includes a non-transitory computer readable medium storing the software instructions that causes a processor to execute the disclosed steps associated with implementations of computer-based algorithms, processes, methods, or other instructions. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges among devices can be conducted over a packet-switched network, the Internet, LAN, WAN, VPN, or other type of packet switched network; a circuit switched network; cell switched network; or other type of network.

Identification of expression level can be performed by any known art method. Preferred methods include quantitative RNA (hnRNA or mRNA) analysis and/or quantitative proteomics analysis. Most typically, but not necessarily, the threshold level for inclusion of neoepitopes will be an expression level of at least 20%, and more typically at least 50% as compared to matched normal, thus ensuring that the epitope is at least potentially ‘visible’ to the immune system. Thus, it is generally preferred that the omics analysis also includes an analysis of gene expression (transcriptomic analysis) to so help identify the level of expression for the gene with a mutation. Viewed from another perspective, transcriptomic analysis may be suitable (alone or in combination with genomic analysis) to identify and quantify genes having a cancer and patient specific mutation. There are numerous methods of transcriptomic analysis know in the art, and all of the known methods are deemed suitable for use herein. Taken the above into consideration, it should therefore be appreciated that a patient sample comprising DNA and RNA from tumor and matched normal tissue can be used to identify specific mutations and to quantify such mutations.

Neoepitopes obtained as described above may be subject to further detailed analysis and filtering using predefined structural and expression parameters, and/or sub-cellular location parameters. For example, it should be appreciated that neoepitope sequences are only retained provided they will meet a predefined expression threshold (e.g., at least 20%, 30%, 40%, 50%, or higher expression as compared to normal), or are identified as having a membrane associated location (e.g., are located at the outside of a cell membrane of a cell). Further contemplated analyses will include structural calculations that delineate whether or not a neoepitope is likely to be solvent exposed, presents a structurally stable epitope, etc. Further examples, methods, and neoepitopes are found in co-pending, International applications PCT/US16/26798 (filed Apr. 8, 2016) and PCT/US16/29244 (filed Apr. 25, 2016), both incorporated by reference herein.

Immunotherapy treatment success requires neoepitopes to be presented via the major histocompatibility complex (MHC complex). Thus, it should be appreciated that the neoepitopes or their precursors must not only be suitable for intracellular processing via appropriate mechanisms (e.g., proteasomal cleavage, formation of a TAP (transporter associated with antigen processing) complex, vesicular transport, etc.) but also have a minimum affinity to the subject's human leukocyte antigen type (HLA-type). Therefore, it is generally preferred that the HLA-type of the subject be determined, either using conventional wet-lab methods, or via in silico methods as further described in more detail below. Viewed from a different perspective, it should be appreciated that identified neoepitopes may be further qualified for prediction of treatment outcome by ascertaining their binding to the patient specific MHC-type.

HLA determination can be performed using various methods in wet-chemistry that are well known in the art, and all of these methods are deemed suitable for use herein. However, in especially preferred methods, the HLA-type can also be predicted from omics data in silico using a reference sequence containing most or all of the known and/or common HLA-types as is shown in more detail below. In short, a patient's HLA-type is ascertained (using wet chemistry or in silico determination), and a structural solution for the HLA-type is calculated or obtained from a database, which is then used as a docking model in silico to determine binding affinity of the neoepitope to the HLA structural solution. Suitable in silico prediction methods of the HLA-type of a patient especially include those described in co-pending, U.S. provisional applications 62/209858 (filed Aug. 25, 2015), which is incorporated by reference herein. Suitable systems for determination of binding affinities include the NetMHC platform (see e.g., Nucleic Acids Res. Jul. 1, 2008; 36(Web Server issue): W509-W512.). Neoepitopes with high affinity (e.g., less than 100 nM, less than 75 nM, less than 50 nM) against the previously determined HLA-type are then selected.

Once subject and tumor specific neoepitopes and HLA-type are identified, computational analysis can be performed by docking neoepitopes to the HLA and determining best binders (e.g., lowest KD, for example, less than 50 nM). It should be appreciated that such an approach will not only predict microsatellite instable (MSI) cancers, but also identify neoepitopes that are most likely to be presented on a cell and therefore most likely to elicit an immune response with therapeutic effect. Of course, it should also be appreciated that such identified HLA-matched neoepitopes can be biochemically validated in vitro.

All preceding steps are performed in silico. Next steps include actual therapy creation (e.g., viral, bacterial, or yeast expression system) for in vivo expression of neoepitopes, and/or in vitro generation of synthetic peptides having neoepitope sequences. Preceding steps will likely provide more than one, more typically more than ten, or more than 100 candidate neoepitopes and suitable choices to be validated.

### Identification of Autoantigens

An autoantigen is an endogenous epitope present in the subject that a healthy subject does not recognize as self. Autoantigens include proteins, nucleic acids, lipids, sugars, and tissue substrates such as collagen or ground substance, that were previously not targeted by the subject immune system, but that become targeted as if they are non-self. This can be caused by a disease process, e.g., caused by a cross-reactive infectious agent.

Infectious agents known to trigger cytokine cascade include, for example, Ebola virus, certain strains of influenza, e.g., 2009 H1N1 (pH1N1) influenza and avian H5N1 influenza virus infection. Other infectious diseases that are known to be capable of causing cytokine cascade include severe acute respiratory syndrome (SARS) caused by a member of the coronavirus family, bacterial sepsis, and smallpox.

Autoantigens also include self-epitopes originally present in an immunologically protected anatomical site, e.g., corneal tissue, deep bone tissue, and the like. An autoantigen may include a “self” antigen to which the subject would normally react in a beneficial manner, for example, T cell anergy, regulatory T cells, Thl, or Th2 cells. Such a site can be exposed during the lifetime of the subject by an injury, infectious process and/or surgical procedure.

Autoantigens can be identified by a number of methods, including , ELISA tests using patient serum screened against a collection of human proteins. For example, autoantigen discovery may be performed using a synthetic human peptidome (see e.g., Nat Biotechnol. 2011, 29(6):535-41) or human proteome (e.g., PLoS ONE 10(5): e0126643;1 Genomics Proteomics Bioinformatics. 2015 August; 13(4):210-8), or customized proteome platforms (e.g., protein array from ADi, Inc; URL: www dotantigendiscovery dotcom). Upon identification of the peptide or protein, further epitope mapping can be performed to identify the autoreactive portion using methods well known in the art.

In addition, there are numerous known autoantigens that are also deemed suitable for use in conjunction with the teachings presented herein. For example, suitable known autoantigens include those targeted and/or precipitated by antinuclear antibodies, antineutrophil cytoplasmic antibodies (ANCA), anti-double stranded DNA (anti-dsDNA), anticentromere antibodies (ACA), antihistone antibodies, cyclic citrullinated peptide antibodies (CCP), rheumatoid factor, and selected proteins such as intrinsic factor, tissue transglutaminase, beta 2 glycoprotein, etc.

### Identification Of Exogenous Antigens

An exogenous antigen is, for example, a food antigen, such as nut allergens, strawberry allergens and the like. Food allergens include, e.g., one or more of a gliadin, a gluten, and a peanut allergen, to name but a few art known food allergens. All of these are deemed suitable for use in conjunction with the teachings herein. Most typically, tests for proper selection of the food antigen will typically involve ELISA-based testing using patient serum against a collection of food antigens, which may be highly purified and isolated (e.g., gluten, gliadin, peanut allergen) or be present as complex mixture (e.g., using food intolerance tests by Biomerica).

For the purpose of the present invention, an exogenous antigen is also considered to include other exogenous substances that may be introduced into a subject to which some subjects may react with a cytokine storm, such as oral medicaments, including drugs, hormones, excipients, topical creams, ointments and the like, injectable medicaments, including proteins, hormones and antibodies, e.g., immune serums, implantable materials such as controlled release hormones and other medicaments, surgical materials, and the like.

### Peptides and Antibodies

To obtain the epitope(s) identified above, it is contemplated that the epitope(s) identified above are prepared in vitro to yield a synthetic epitope, e.g., a synthetic peptide. There are numerous methods known in the art to prepare synthetic peptides, and all known methods are deemed suitable for use herein. For example, peptides with cancer neoepitope sequences can be prepared on a solid phase (e.g., using Merrified synthesis), via liquid phase synthesis, or from smaller peptide fragments. In some other aspects, peptides could also be produced by expression of a recombinant nucleic acid in a suitable host (especially where multiple neoepitopes are on a single peptide chain, optionally with spacers between neoepitopes or cleavage sites).

Therefore, the structure of the synthetic peptides corresponding to the neoepitope sequences may be X-L₁-(Aₙ-L₂)ₘ-Q, in which X is an optional coupling group or moiety that is suitable to covalently or non-covalently attach the synthetic peptide to a solid phase, L₁ is an optional linker that covalently links the synthetic peptide to a solid phase, or to the coupling group. Aₙ is the synthetic peptide having the neoepitope sequence, with A being a natural (proteinogenic) amino acid and n is an integer between 7 and 30, and most typically between 7 and 11 or 15-25. L₂ is an optional linker that may be present, especially where multiple synthetic peptide sequences (identical or different) are in the construct, and m is an integer, typically between 1 and 30, and most typically between 2 and 15. Finally, Q is a terminal group which may be used to couple the end of the synthetic peptide to the solid phase (e.g., to sterically constrain the peptide) or to a reporter group (e.g., fluorescence marker) or other functional moiety (e.g., an affinity marker and/or an immune modulator molecule according to the present invention). Consequently, it should be noted that where the synthetic peptide is used for direct MHC-I binding, the overall length will be between 8 and 10 amino acids. Similarly, where the synthetic peptide is used for direct MHC-II binding, the overall length will be between 14 and 20 amino acids. On the other hand, where the synthetic peptide is processed in the cell (typically via proteasome processing) prior to MHC presentation, the overall length will typically be between 10 and 40 amino acids, with the changed amino at or near a central position in the synthetic peptide.

For example, X could be a non-covalent affinity moiety (e.g., biotin) that binds a corresponding binding agent (e.g., avidin) on the solid phase, or a chemical group (with or without spacer) that reacts with the N- or C-terminal amino or carboxyl group of the peptide, or a selectively reactive group (e.g., iodoacetyl or maleimide group) that reacts with a sulfhydryl group in the peptide or linker L₁. L₁ may be used to increase the distance of the synthetic peptide from the solid phase and will therefore typically comprise a flexible linear moiety (e.g., comprising glycol groups, alkoxy groups, glycine, etc.) having a length of equivalent to between about 2-20 carbon-carbon bonds (e.g., between 0.3 nm and 3 nm). Of course, it should also be appreciated that the synthetic peptide may use the solid phase on which the peptide was produced and as such not require a separate coupling group or linker.

Depending on the particular synthetic peptide and coupling method, it should be appreciated that the nature of the solid phase may vary considerably, and all known solid phases for attachment of peptides are deemed suitable for use herein. For example, suitable solid phases include agarose beads, polymer beads (colored or otherwise individually addressable), wall surfaces of a well in a microtiter plate, paper, nitrocellulose, glass, etc. The person of ordinary skill in the art will be readily appraised of a suitable choice of solid phase and attachment chemistry. In further preferred aspects, it is also noted that the solid phase will generally be suitable for protocols associated with phage display methods such as to allow peptides presented on a phage (or other scaffold carrier) to reversibly bind to the solid phase via the synthetic peptide. In still further contemplated uses, it should also be recognized that the solid phase may be a carrier protein used in vaccination (e.g., albumin, KLH, tetanus toxoid, diphtheria toxin, etc.), particularly where the synthetic protein is used as a vaccine in a mammal or as an immunogenic compound in a non-human mammal for antibody production. Likewise, the synthetic protein may also be used as a vaccine or immunogenic compound without any carrier.

To obtain an antibody that binds to the identified neoepitope, the above described synthetic peptide is employed as an antigen, with a suitable adjuvant and/or an expression vector encoding a multi epitope construct, to elicit an immune response in a suitable subject human or other mammalian immunological system.

### Preparation of Chimeric Immune Modulators

The inventive immune modulator moieties are prepared by any art known method. For example, a peptide epitope is contemplated to be prepared by synthesizing an epitope as described hereinabove and covalently linking the peptide epitope to a preferred immune modulator, e.g., via the Q moiety. Linkers according to the invention are selected to join a peptide that provides an epitope of interest, according to the invention, and one or more immune modulator molecules, while preserving the antigenic specificity of the epitope and preserving the ability of the immune modulator molecule to downregulate an immune response and/or as an anti-inflammatory molecules.

Alternatively, the peptide epitope can be extended to include a peptide linker that from about 1 to about 15 amino acid residues in length, followed by a peptide immune modulating molecule, such as an anti-inflammatory cytokine. Peptide spacers can be selected as described, by for example, U.S. Pat. No. 6,783,761, including, for example, Gly-Pro-Ser-Leu (SEQ ID NO: 1) and/or Ser-Ser-Gly-Pro-Ser- (SEQ ID NO: 2). Most typically, linkers will be selected to have substantial flexibility and lack immunogenicity, including (G4S)3, G8, G6, (EAAAK)1-3, etc. Further suitable linkers can be selected following well known considerations (e.g., Adv Drug Deliv Rev. 2013 Oct. 15; 65(10): 1357-1369).

Linkers for connecting the immune modulator(s) molecules with the epitope of interest include peptide linkers, polymer linkers and/or a direct, covalent bond between the immune modulator component and the epitope.

In certain embodiments, where both components, and the linkers are peptides, the chimeric immune modulator is optionally encoded by a nucleic acid expression vector, that when expressed in a suitable host cell, will produce a single expressed chimeric fusion protein. Suitable expression vectors include plasmid and/or virus vectors, depending on the selected host cell expression system. Other suitable protein expression vectors known in the art may be selected based upon the expression host (e.g., an expression vector with a mammalian promoter system would be suitable for expression in a human cell line whereas a yeast or bacterial expression plasmid would be selected if expression in either of these organisms was desired.

The invention also encompasses a bacterial or yeast protein expression system comprising a bacterial or yeast cell transformed with a plasmid construct comprising a nucleotide sequence that encodes the inventive chimeric immune modulator , as described in any of the preceding paragraphs. Suitable bacterial strains include, for example, Escherichia coli. Suitable yeast strains include, for example, Pichia pastoris. In still further contemplated aspects, the linker may also be a non-peptide entity, and will preferably include a relatively short and flexible polar polymeric chain. For example, suitable linkers include polyalkylene ethers or glycols having between 2-100 repeat units. Alternatively, the linker may also be a polysaccharide linker, or linking may be achieved by chemical modification of at least one of the moieties to then facilitate covalent bonds between the moieties.

### Treatment Methods

The invention provides methods of modulating an over-reaction of a subject's immune system to an epitope, the methods including administering an effective amount of the inventive chimeric immune modulator to a subject experiencing an immune system over-reaction caused by exposure to an identified epitope.

The invention also provides a method of treating cancer in a subject diagnosed with a cancer comprising:
a) administering an amount of a neoepitope-based anticancer therapy effective to stimulate an immune response to the subject; and
b) administering an effective amount of the chimeric immune modulator of claim 1 to the subject, wherein the epitope of the chimeric immune modulator is the neoepitope of a).

In an alternative embodiment, the neoepitope-based therapy, e.g., an anticancer therapy of a) comprises administering a vaccine to the subject, wherein the vaccine comprises a nucleic acid that encodes the neoepitope, a protein-based vaccine comprising the neoepitope, a cell-based vaccine that presents the neoepitope to the immune system of the subject, and a viral expression system-based vaccine that presents the neoepitope to the subject's immune system.

Where the inventive chimeric immune modulator is a peptide based construct, it is also contemplated to deliver the chimeric immune modulator to a patient in the form of an expression vector that will express the chimeric immune modulator in situ, in the tissues of the subject. Likewise, the chimeric immune modulator may also be co-expressed in a dendritic cell that expresses one or more recombinant neoepitopes or autoantigens to so provide contextual information to the T-cells.

The inventive method optionally also includes a step of administering an antibody against the neoepitope to a subject having an over-reaction of the immune system. For example, the antibody is an immunoglobulin that does not activate a cellular immune response in the subject.

This is, for example, an antibody with an IgG4 portion having, for example, an inactive Fc domain.

The inventive method also optionally includes a step of administering a therapeutically active molecule to a subject having an over-reaction of the immune system, wherein the therapeutically active molecule reduces epitope concentrations (or exposure of the epitope to immune competent cells) in the subject. The therapeutically active molecule is, for example, an antibody that binds to a virus that expresses the neoepitope in the subject.

In certain embodiments, the over-reaction of the immune system is associated with an MHC-I and/or MHC-II polymorphism. The MHC-II polymorphism can result in one or more of Type-I diabetes, rheumatoid arthritis, celiac disease, and pemphigus vulgaris. The MHC-I polymorphism can result in one or more of Hashimoto's Thyroiditis, systemic lupus erythematodes, and blepharitis.

The invention also provides methods of using the chimeric immune modulator to modulate, e.g., downregulate or interrupt an over-reaction of a subject's immune system, e.g., as a cytokine storm, the method comprising administering an effective amount of the inventive chimeric immune modulator to the subject, optionally in the form of a pharmaceutical composition.

A pharmaceutical composition, for example, includes the chimeric immune modulator dissolved or suspended in an aqueous carrier that is physiologically suitable for intravenous, intramuscular and/or subcutaneous injection, infusion, nasal spray, topical administration and/or pulmonary inhalation into the subject.

When the chimeric immune modulator is suitable for oral ingestion and absorption, the carrier can be any standard oral excipient or formulation, or when required, a carrier that is effective to enhance oral absorption of a macromolecule. These include, for example, permeation enhancers, mucoadhesive polymeric systems, enzyme inhibitors and the like. The state of the art of strategies for oral administration of macromolecules is reviewed by Park et al., 2011, Reactive & Functional Polymers 71: 280-287, Muheem et al., 2016, Saudi Pharmaceutical Journal, 2016 24: 413-428. Available oral absorption enhancing technologies include Eligen® from Emisphere Technologies (USA) (or Elis), and Oral-Lyn: Generex Biotechnology Corp. (Canada) (buccal absorption).

An effective amount of the chimeric immune modulator for the above methods is readily determined by the artisan. The artisan can readily optimize the dose by administering the inventive chimeric immune modulator and titrating the dose and amount until an ongoing cytokine storm is modulated, inhibited and/or aborted. The dose is repeated as needed until the underlying cytokine reaction abates and/or until the subject is out of danger. The timing of the doses can be determined in one of several ways. For example, an effective amount can be administered a metronomic schedule. The metronomic schedule is optionally based on the concentration of the administered immune modulator, a time schedule, or based on severity of the over-reaction of the subject's immune system.

In addition, the step of administering includes administering the chimeric immune modulator to the subject topically, by intravenous, intramuscular and/or subcutaneous injection, infusion, nasal spray, topical administration and/or pulmonary inhalation intravenously, subcutaneously, or orally.

In a further embodiment, the inventive method includes the additional step of administering an antibody that binds to the epitope, thus reducing the body burden of the epitope, while simultaneously modulating the proinflammatory reaction. The antibody is preferably one that does not activate a cellular immune response. More preferably, the antibody is an IgG4 portion having an Fc domain engineered to prevent the antibody from activating cellular immune responses and curtail FcR-mediated processes. See, e.g., Slauthhour et al, 2016, Protein Engineering, Design and Selection doi: 10.1093/protein/gzw040

In an alternative embodiment, the inventive method includes the step of administering a therapeutically active molecule that inhibits or downregulates a system that delivers the epitope to the subject. The therapeutically active molecule is preferably an antibody, which will bind to a virus that delivers the epitope to the subject.

The inventive method also optionally includes administering an effective amount of an immunosuppressive drug, such as a glucorticosteroid, ribavirin, an anti-MHC II antibody, an anti-cytokine antibody, cyclosporine, cyclophosphamide, and any other art known immunosuppressant.

The invention method also provides for measuring a metric of an immune response to a treatment associated with the chimeric immune modulator. The metric provides a quantitative basis for decisions regarding intervention in the treatment of the subject. The metric can include at least one of:
a) a measurement of at least one cytokine;
b) a measurement of a T-cell concentration;
c) a measurement of a T-cell frequency;
d) an ELISA measurement of B-cell activation; and
e) a measurement of T-cell proliferation.

The metric to be measured includes, for example:
a) a measurement of at least one cytokine;
b) a measurement of a T-cell concentration;
c) a measurement of a T-cell frequency;
d) an ELISA measurement of B-cell activation; and
e) a measurement of T-cell proliferation.

Thus, the invention also includes determining an intervention point based on the metric, and measuring a metric of an immune response to a treatment associated with the epitope.

### EXAMPLES

The following examples are provided in order to illustrate the present invention, without intending to limit the scope of the present invention.

### Example 1

### Preparing a Chimeric Immune Modulator

A. Identifying a Cancer Neoepitope and Preparing an Anticancer Vaccine

A subject is diagnosed with a melanoma. As much of the melanoma tumor or tumors as possible is removed surgically. DNA is extracted from a sample of the removed tumor tissue, and the extracted DNA is subjected to genomic sequencing, and compared to genomic sequencing of DNA extracted from the subject's normal tissue. Alternatively, the genomic DNA of the subject's tumor is compared to a database of human genomic sequences. The analysis is performed in silico by location-guided synchronous alignment of tumor and normal samples as, for example, disclosed in US20120059670A1 and US 20120066001A1 using BAM files and BAM servers.

Based on the genomic analysis, one or more non-silent mutations are identified, and screened by the methods described, for instance, in the example section of US20160008447. The identified neoepitopes are also HLA matched, against the subject's tissues, or in silico.

An anticancer vaccine that includes one or more identified polypeptide neoepitope unique to the melanoma is prepared and administered to the subject in order to induce in the subject an endogenous reaction targeted against the melanoma.

B. Preparing a Chimeric Immune Modulator

Against the possibility that the vaccination of the subject against the melanoma tumor producted a life threatening overreaction, such as a cytokine storm, a chimeric immune modulator is prepared.

The immune modulator is prepared by linking the above described neoepitope employed to prepare the anticancer vaccine with an immune modulator, such as IL-4, IL-10, IL-11, IL-12, and IL-17 or to a V-domain immunoglobulin (Ig)-containing suppressors of T-cell activation (VISTA).

C. Administering the Immune Checkpoint Inhibitor

If the subject vaccination with the anti-melanoma anticancer vaccine has a life-threatening exaggerated reaction to the course of treatment after vaccination, e.g., a cytokine storm, the personalized immune checkpoint inhibitor is administered intravenously as a rescue treatment, in order to downregulate the over-reaction and prevent nonspecific organ damage and possibly death from the over-reaction.

The immune point checkpoint inhibitor is administered in an amount, at a rate, and for a duration that is determined to be clinically necessary by the artisan supervising the anticancer treatment.

1. A chimeric immune modulator comprising a first immune modulating portion and a second portion, wherein the first portion comprises a ligand for an immune checkpoint inhibition receptor; wherein the second portion comprises an epitope for which there is knowledge that a percentage of subjects to which the epitope is administered is at risk for developing an immunological over-reaction; and wherein the modulating portion and the second portion are coupled together via a linker.

2. The chimeric immune modulator of claim 1, wherein the epitope is a neoepitope, an autoantigen and/or an exogenous antigen.

3. The chimeric immune modulator of claim 1, wherein the linker is a peptide, a polymer, or a covalent bond.

4. The chimeric immune modulator of claim 1, wherein the ligand is one or more of a CTLA4 ligand, a PD1 ligand, and a B7-H4 molecule.

5. The chimeric immune modulator of claim 4, wherein the ligand is a CTLA4 ligand or a PD1 ligand.

6. The chimeric immune modulator of claim 1, wherein the modulating portion further comprises a negative checkpoint regulator, or an inhibitor of a co-stimulator molecule.

7. The chimeric immune modulator of claim 6, wherein the modulating portion comprises a negative checkpoint regulator.

8. The chimeric immune modulator of claim 6, wherein the modulating portion comprises an inhibitor of a co-stimulatory molecule.

9. The chimeric immune modulator of claim 8, wherein the co-stimulatory molecule is one or more of CD80, and CD86.

10. The chimeric immune modulator of claim 1, wherein the epitope is patient-specific and/or is tumor-specific.

11. The chimeric immune modulator of claim 1, wherein the epitope is an HLA-matched neoepitope.

12. The chimeric immune modulator of claim 1, wherein the chimeric immune modulator comprises multiple distinct epitopes.

13. A method of modulating an over-reaction of a subject's immune system to an epitope, the method comprising administering an effective amount of the chimeric immune modulator of claim 1 to the subject, and wherein the epitope is optionally an autoantigen, a neoantigen, or an exogenous antigen.

14. (canceled).

15. The method of claim 13, further comprising a step of administering an antibody that binds to the epitope, wherein the antibody is an immunoglobulin that does not activate a cellular immune response.

16-26. (canceled)

27. A method of treating cancer in a subject diagnosed with a cancer comprising:
a) administering an amount of a neoepitope-based anticancer therapy having an amount of a neoepitope effective to stimulate an immune response to the subject; and
b) administering an effective amount of the chimeric immune modulator of claim 1 to the subject, wherein the epitope of the chimeric immune modulator is the neoepitope of a).

28. The method of claim 27, wherein the neoepitope-based therapy of a) comprises administering a vaccine composition to the subject, wherein the vaccine composition comprises a nucleic acid that encodes the neoepitope, a protein-based vaccine comprising the neoepitope, a cell-based vaccine that presents the neoepitope the immune system of the subject, and a viral expression system-based vaccine that presents the neoepitope to the immune system of the subject.

29-44. (canceled)

45. A method of modulating an over-reaction of an immune system to an exogenous antigen comprising administering an effective amount of the chimeric immune modulator of claim 1, wherein the epitope is the exogenous antigen is an influenza virus, a SARS virus, a bacterium causing sepsis or a smallpox virus.

46. The method of claim 45, wherein the exogenous antigen is a food antigen that triggers the over-reaction.

47. (canceled)

48. A method of preparing the chimeric immune modulator of claim 1, comprising covalently linking one or more epitopes that cause or may cause an over-reaction of a subject's immune system with one or more immune modulators that will downregulate a cytokine storm.

49-50. (canceled)

51. The chimeric immune modulator of claim 7, wherein the negative checkpoint regulator is a VISTA molecule.
